(19) United States
(12) Patent Application Publication
(10) Pub. No.: US 2007/0149864 A1
(43) Pub. Date: Jun. 28, 2007

(54) Monitoring device for multiple tissue sites

(76) Inventor: Marko Laakkonen, Vantaa (FI)

Correspondence Address: ANDRUS, SCEALES, STARKE & SAWALL, LLP, 100 EAST WISCONSIN AVENUE, SUITE 1100, MILWAUKEE, WI, 53202, US

(21) Appl. No.: 11/319,014

(22) Filed: Dec. 27, 2005

(51) Int. Cl. A61B 5/00

(52) U.S. Cl. 600/310; 600/323

(57) ABSTRACT

The invention relates to a patient monitoring device, especially to a pulse oximeter, provided with multiple sensors for performing simultaneous measurements at multiple tissue sites. In order to reduce the hardware required by the measurement, a repeating drive pulse sequence is generated, which contains drive pulses for the emitter elements of the plurality of sensors. Furthermore, each drive pulse of the sequence is supplied to a corresponding emitter element and sensor-specific detectors connected in parallel are employed to produce an electric reception signal received at the monitoring device.

### FIELD OF THE INVENTION

The present invention relates generally to monitoring devices intended for monitoring the attenuation of light in a subject. More particularly, the present invention relates to a monitoring device that provides simultaneous measurement results from multiple tissue sites of the subject. The monitoring device is typically a pulse oximeter.

### BACKGROUND OF THE INVENTION

Pulse oximetry is at present the standard of care for continuous monitoring of arterial oxygen saturation (SpO₂). Pulse oximeters provide instantaneous in-vivo measurements of arterial oxygenation, and thereby an early warning of arterial hypoxemia, for example.

A pulse oximeter comprises a computerized measuring unit and a sensor attached to the patient, typically to a finger or ear lobe. The sensor includes a light source for sending an optical signal through the tissue and a photo detector for receiving the signal after transmission through the tissue. On the basis of the transmitted and received signals, light absorption by the tissue can be determined. During each cardiac cycle, light absorption by the tissue varies cyclically. During the diastolic phase, absorption is caused by venous blood, tissue, bone, and pigments, whereas during the systolic phase there is an increase in absorption, which is caused by the influx of arterial blood into the tissue. Pulse oximeters focus the measurement on this arterial blood portion by determining the difference between the peak absorption during the systolic phase and the constant absorption during the diastolic phase. Pulse oximetry is thus based on the assumption that the pulsatile component of the absorption is due to arterial blood.

Light transmission through an ideal absorbing sample is determined by the known Lambert-Beer equation as follows:
Iₒᵤₜ=Iᵢₙe^{-εDC}, (1)

where Iᵢₙ is the light intensity entering the sample, Iₒᵤₜ is the light intensity received from the sample, D is the path length through the sample, ε is the extinction coefficient of the analyte in the sample at a specific wavelength, and C is the concentration of the analyte. When Iᵢₙ, D, and ε are known, and Iₒᵤₜ is measured, the concentration C can be calculated.

In pulse oximetry, in order to distinguish between two species of hemoglobin, oxyhemoglobin (HbO₂), and deoxyhemoglobin (RHb), absorption must be measured at two different wavelengths, i.e. the sensor normally includes two different light emitting diodes (LEDs). The wavelength values widely used are 660 nm (red) and 940 nm (infrared), since the said two species of hemoglobin have substantially different absorption values at these wavelengths. Each LED is illuminated in turn at a frequency which is typically several hundred Hz.

Conventional pulse oximeters are restricted to measurement of arterial oxygen saturation at a single tissue site. Therefore, if continuous and simultaneous oxygen status measurements are needed from several tissue sites, a straightforward method is to use a plurality of conventional pulse oximeters simultaneously. The need may arise, for example, during a delivery when both the mother and the infant need to be monitored simultaneously.

To eliminate the above drawback, pulse oximeters have been developed, which provide simultaneous and continuous measurement results from a plurality of tissue sites. U.S. Pat. No. 6,714,804 discloses a stereo pulse oximeter providing simultaneous and continuous oxygen status measurements at multiple tissue sites. The pulse oximeter is provided with multiple sensors attachable to distinct tissue sites. Each sensor may be connected through a separate cable and sensor interface to a signal processor. Alternatively, a so-called stereo sensor, which is provided with multiple branches, may connect the sensors through a common patient cable to a single connection at the pulse oximeter. From the said single connection each sensor signal is branched off to the respective sensor interface.

U.S. Pat. No. 5,218,962 further discloses a multiple region pulse oximetry probe and oximeter, which enable the blood characteristics to be sensed at two or more unique sites. In one embodiment, the probe housing accommodates probe elements for two distinct tissue regions, but the probe elements may also be mounted in separate probe housings. The oxygen saturation values obtained from two tissue sites are compared with each other to improve the reliability of the measurement.

A drawback related to current pulse oximeters providing simultaneous measurement results from multiple tissue sites is the rather extensive multiplication of the hardware required by the parallel measurements. As mentioned above, each sensor normally requires a dedicated interface that typically includes both signal processing means for the electric signal received from the respective sensor and current drivers for the emitters of the respective sensor.

The present invention seeks to eliminate the above drawbacks and to bring about a novel mechanism for simultaneous non-invasive measurement of blood characteristics at multiple tissue sites.

### SUMMARY OF THE INVENTION

The present invention seeks to provide a cost-effective measurement arrangement for monitoring the attenuation of light at multiple tissue sites.

In the present invention, a monitoring device is operably connected to at least two sensors for simultaneous measurement of the attenuation of light at multiple tissue sites. Each sensor comprises at least one emitter element for emitting radiation and a sensor-specific detector for receiving the radiation and for producing an electric signal in response to the radiation. The sensor-specific detectors are connected in parallel to produce an electric reception signal at a terminal pair common to the sensor-specific detectors. As the drive pulses that activate a particular emitter element are supplied to that emitter element in pre-allocated time slots, the monitoring device knows when a particular detector generates the electric reception signal or which one of the detectors generates the electric reception signal in a particular time window. The electric reception signal may thus be supplied to the monitoring device through a single wire pair, which allows the use of a reception branch of a conventional single-sensor monitor, i.e. no hardware multiplication is necessary on the reception side of the monitoring device for receiving and processing signals from a plurality of sensors. Consequently, the sensors may be connected to the said reception branch through a single patient cable comprising a branch for each sensor and common cable segment comprising the above-mentioned single wire pair.

Thus one aspect of the invention is providing a measurement method for a monitoring device intended for monitoring the attenuation of light in at least one subject, the monitoring device being operably connected to a plurality of sensors, each sensor comprising at least one emitter element for emitting radiation and a sensor-specific detector for receiving the radiation, whereby the monitoring device is operably connected to a plurality of sensor-specific detectors. The method includes the steps of generating a repeating drive pulse sequence containing drive pulses for the emitter elements of the plurality of sensors and supplying each drive pulse of the drive pulse sequence to a corresponding emitter element. The method further includes the steps of employing a parallel connection of the plurality of sensor-specific detectors to produce an electric reception signal at a terminal pair common to the sensor-specific detectors and receiving the electric reception signal at the monitoring device.

Another aspect of the invention is that of providing a monitoring device intended for monitoring the attenuation of light in at least one subject. The measurement arrangement includes a plurality of sensors for a plurality of tissue sites, each sensor comprising at least one emitter element for emitting radiation and a sensor-specific detector for receiving the radiation, wherein the sensor-specific detectors are connected in parallel for producing an electric reception signal at a terminal pair common to the sensor-specific detectors and drive pulse generation means for generating a repeating drive pulse sequence containing drive pulses for the emitter elements of the plurality of sensors. The monitoring device further includes switching means for connecting each drive pulse of the drive pulse sequence to a corresponding emitter element and connection means for connecting the terminal pair operably to the monitoring device, thereby to receive the electric reception signal at the monitoring device.

Since the additional hardware needed due to the multiple sensors may be minimized, the invention provides a cost-effective solution for monitors providing continuous measurement results from multiple tissue sites. This also translates to minimal area/space requirements allowing the implementation of compact size monitors.

A still further aspect of the invention is that of providing a computer program product by means of which an existing monitoring device may be upgraded to carry out a simultaneous measurement from a plurality of sensors. The computer product comprises a first program code portion configured to control the monitoring device to generate a repeating drive pulse sequence containing drive pulses for the emitter elements of the plurality of sensors, a second program code portion configured to connect the drive pulses to respective emitter elements in a predetermined order and a third program code portion configured to associate an electric reception signal with the emitter elements of the plurality of sensors, one emitter element at a time according to the predetermined order.

Other features and advantages of the invention will become apparent by reference to the following detailed description and accompanying drawings.

### BRIEF DESCRIPTION OF THE DRAWINGS

In the following, the invention and its preferred embodiments are described more closely with reference to the examples shown in FIG. 1 to 8 in the appended drawings, wherein:

FIG. 1 illustrates a general measurement arrangement according to the invention;

FIG. 2 illustrates one embodiment of the emitter and detector circuitries of the apparatus of the invention;

FIG. 3 illustrates one embodiment of the basic emitter and detector cycles of the apparatus of the invention;

FIG. 4 illustrates one embodiment of the emitter switching unit of the apparatus of FIG. 2;

FIG. 5 and FIG. 6 illustrate the control of the emitter switching unit of FIG. 4 at two distinct time windows;

FIG. 7 illustrates an embodiment comprising sensors emitting at four different wavelengths; and

FIG. 8 illustrates one embodiment of the monitoring device of the invention, which comprises a common current source for all emitter elements.

### DETAILED DESCRIPTION OF THE INVENTION

A monitoring device of the invention comprises a computerized measuring unit and a plurality of sensors that may be attached to multiple tissue sites. Each sensor includes at least one light source, i.e. emitter element, for sending an optical signal through the tissue and a photo detector for receiving the signal transmitted through of reflected from the tissue. The number of emitter elements in a sensor depends on the application used. Plethysmographic data, for example, may be measured with only one emitter element (one wavelength), but a pulse oximeter typically has two or more emitter elements in each sensor.

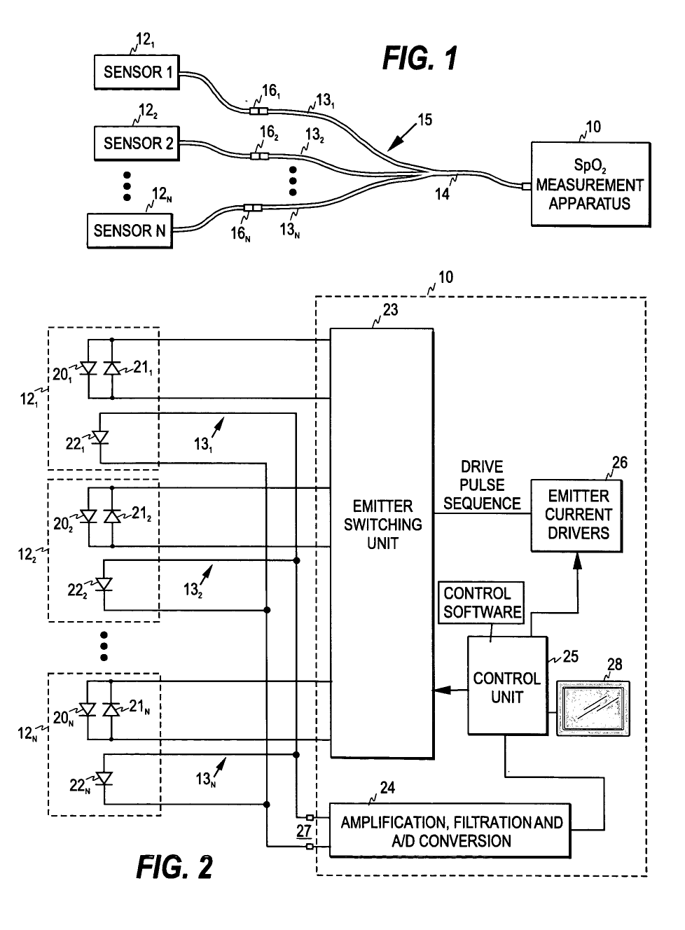

FIG. 1 illustrates one embodiment of the general measurement arrangement of the invention. A single SpO₂ measurement apparatus 10 is provided with N sensors 12₁, to 12_{N} attachable to multiple tissue sites. As noted above, each sensor comprises at least one emitter element for emitting radiation at a minimum of one wavelength and a detector for receiving the radiation and for producing an electric signal in response to the radiation. The detectors may be standard multiwavelength detectors. A single measurement cable 15, comprising N branches, connects the detector of each sensor to the monitoring device. The measurement cable resembles a Y-shaped cable, but instead of the two oblique branches of a Y it comprises N branches 13ᵢ (where N≧2 and i=1,2, . . . , N). A common cable segment 14 connects each branch to the monitoring device. Each sensor may be provided with a connector 16ᵢ (i=1 . . . N) for connecting the sensor to a mating connector mounted to the end of a respective branch of the measurement cable. In one embodiment of the invention, the mating connectors may be different from each other to allow the use of different sensors or sensors of different manufacturers.

FIG. 2 illustrates one embodiment of the emitter and detector circuitries of the invention. In this example, each sensor 12ᵢ comprises two LEDs 20ᵢ and 21ᵢ (i=1, . . . , N) connected in parallel and back-to-back, i.e. in each sensor the anode of the first LED and the cathode of the second LED are connected together and form a first common pole, while the cathode of the first LED and the anode of the second LED are connected together to form a second common pole. Each sensor further comprises a sensor-specific photodetector 22ᵢ, which receives the light transmitted by the LEDs of the sensor and propagated through or reflected from the tissue and converts the optical signal into an electric signal. The sensor-specific detectors are connected in parallel, and the said electric signal is supplied to the monitoring device through a single anode/cathode wire pair, i.e. through the common cable segment 14 of FIG. 1. The detectors are thus connected to the monitoring device through a single terminal pair 27, i.e. similarly as the sensor is connected to the monitoring device in single-sensor monitoring device. Therefore, the reception branch 24 of the monitoring device may be implemented similarly as in a single-sensor monitoring device. The reception branch typically comprises an input amplifier, a band-pass filter, and an A/D converter. The digitized signal output from the A/D converter is supplied to a control unit 25, which processes the signal data and displays the analysis results on the screen of a display unit 28. The control unit is provided with control software that controls the activation of the LEDs in the sensors. Therefore, the control unit also knows from which one of the detectors the signal data originates in each time window.

The control unit controls an emitter current source 26 to generate a drive pulse sequence, which contains drive pulses for each emitter element in pre-allocated time windows. The drive pulse sequence is supplied to an emitter switching unit 23. The control unit controls the switches of the switching unit so that each drive pulse of the drive pulse sequence is supplied to the corresponding emitter element.

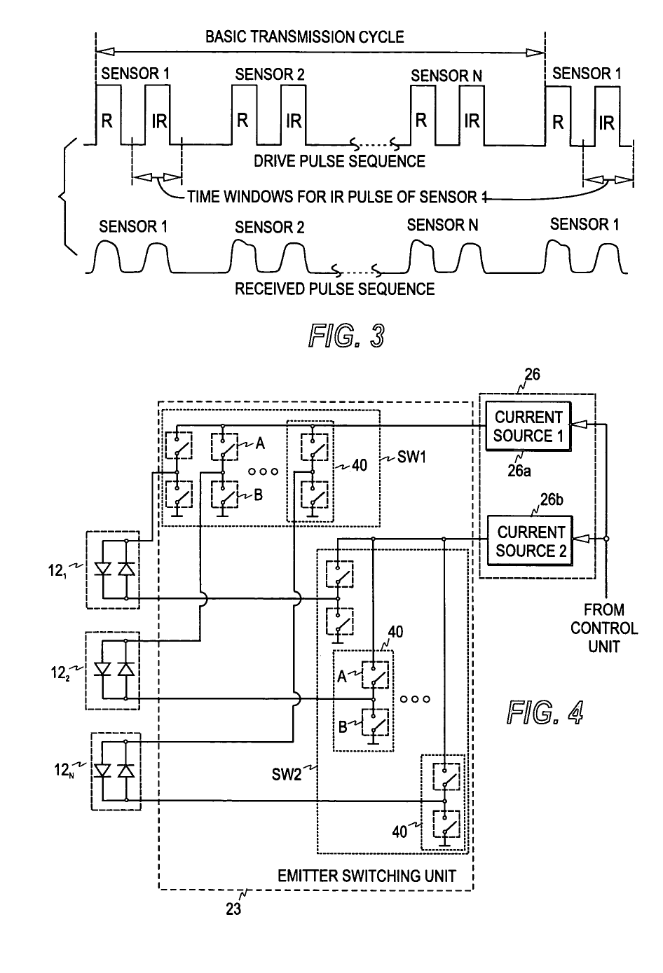

FIG. 3 illustrates one embodiment of the repeating drive pulse sequence generated by the emitter current source and the corresponding detector signal received by the monitoring device. As noted above, each drive pulse has a pre-allocated time window within the drive pulse sequence. In other words, since the reception of the pulses is performed through a single wire pair, the drive pulses are time-multiplexed into the sequence and they do not overlap. Generally, the order of the drive pulses (i.e. the pulse pattern) may be arbitrary within the drive pulse sequence, but it is preferable that the two drive pulses of an individual sensor are after each other so that the time difference between the red and infrared pulses is minimized for each sensor. In the embodiment of FIG. 3, the sensors appear in order, i.e. each transmission cycle starts with the two drive pulses of the first sensor and ends after the two drive pulses of the N^{th} sensor. Any known sequential control technique may, however, be used to drive the red and infrared emitter elements. For example, the pulse pattern may change as a function of time.

FIG. 4 illustrates one embodiment of the emitter current source 26 and the emitter switching unit 23. For reasons of clarity, the detectors have been omitted in the figure. The emitter current source comprises two current sources 26a and 26b, which output the pulse sequence of FIG. 3. In this embodiment, one of the current sources generates the pulses of all red emitter elements in the sensors, while the other current source generates the pulses of all infrared emitter elements in the sensors. Therefore, the first current source 26a is connected to the anodes of the emitter elements of the first type (red or infrared), while the second current source 26b is connected to the anodes of the emitter elements of the second type (infrared or red). The connection is formed through the emitter switching unit, which comprises 2N switching units 40, each of which comprises a first switching element A and a second switching element B connected in series.

The switching units may be divided into two groups: a first group SW1 comprising N units and a second group SW2 also comprising N units. The switching units of the first group switch the drive pulses output from the first current source to the emitter elements of the first type, while the switching units of the second group switch the drive pulses output from the second current source to the emitter elements of the second type. In each group, the first terminal of all switching elements A is connected to the output of the respective current source. The second terminal of all switching elements A is in turn connected to the anode of the emitter element driven by the respective current source. In each switching unit, the said second terminal is further connected to the first terminal of the second switching element B and the second terminal of the second switching element is connected to ground. The second terminal of the first switching elements and the first terminal of the second switching elements thus form a common pole P, which is connected to the anode of the emitter element driven by the respective current source and which may also be connected to ground through the respective second switching element. The first switching elements operate as drive switches which connect each current pulse to the correct emitter element, while the second switching elements operate as current sink switches.

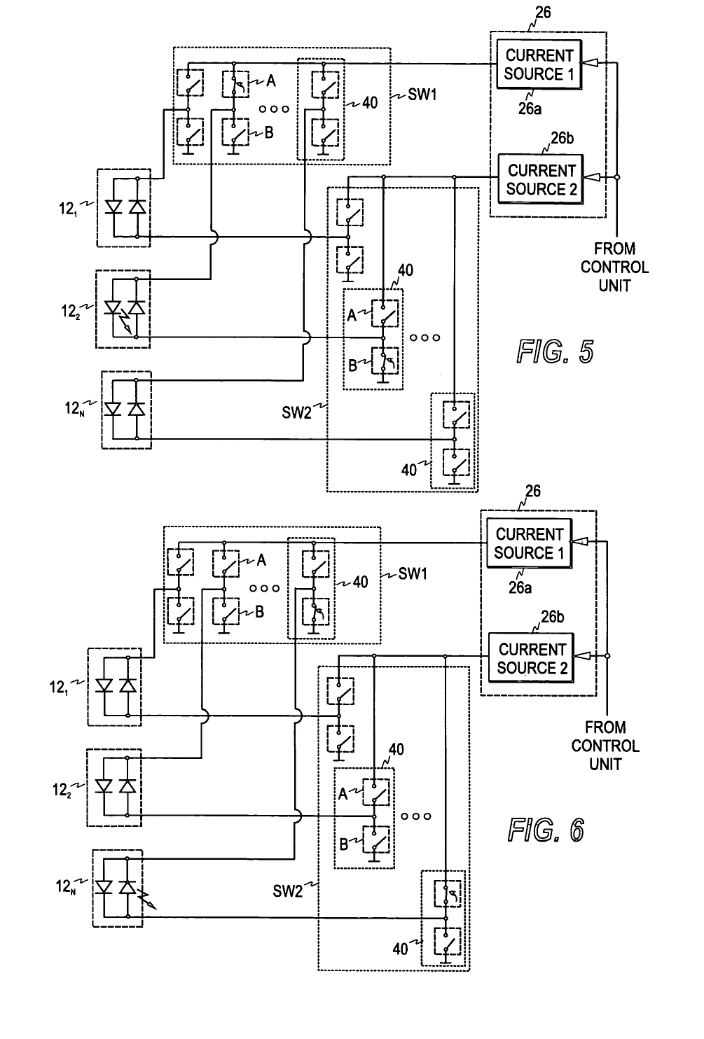

The control unit controls the switching elements so that when the drive pulse of the i^{th} emitter element of the first type is output, switching element A in the i^{th} switching unit of the first group and switching element B in the i^{th} switching unit of the second group are closed (on). The other switching elements remain open (off). Correspondingly, when the drive pulse of the i^{th} emitter element of the second type is output, switching element A in the i^{th} switching unit of the second group and switching element B in the i^{th} switching unit of the first group are closed (on). FIG. 5 and 6 illustrate the control of the switching elements by showing the switches when the drive pulses of the second emitter element of the first type is output (FIG. 5) and when the drive pulses of the N^{th} emitter element of the second type is output (FIG. 6).

The two current sources and the emitter switching unit enable current to be supplied through the sensors in both directions. In each time window corresponding to a drive pulse the control unit thus closes two of the switching elements, the said two elements being selected in accordance with the emitter element to which the time window is allocated.

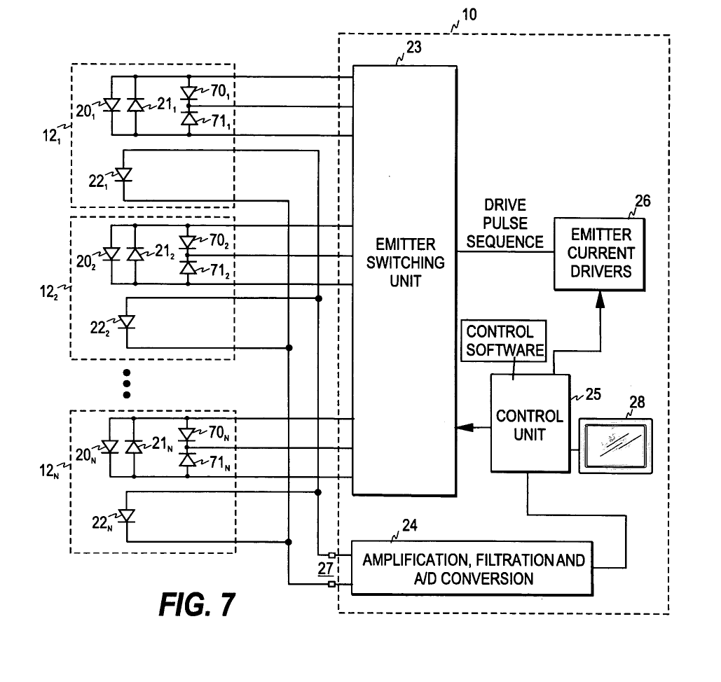

As noted above, each sensor may be provided with one or more emitter elements. FIG. 7 illustrates an embodiment of the invention, in which each sensor comprises four emitter elements emitting, respectively, at four wavelengths. In comparison with the embodiment of FIG. 2, each sensor now further includes a third LED 70ᵢ and a fourth LED 71ᵢ (i=1, 2, . . . N). In each sensor, the anode of the third LED 70ᵢ is connected to the terminal formed by the anode of the first LED and the cathode of the second LED and the anode of the fourth LED 71ᵢ is connected to the terminal formed by the cathode of the first LED and the anode of the second LED. The cathodes of the third and fourth LEDs are connected together. In comparison with the embodiment of FIG. 4, the embodiment of FIG. 7 does not require additional switching elements, since each third and fourth LED may still be driven through one of switching units in the first and second groups, respectively, and the common cathode of each LED pair may be connected to ground through one of the switching elements shown in FIG. 4, so that each LED may be illuminated in a dedicated time window of the drive pulse sequence.

Although the sensors are typically similar in regard to the wavelengths used, it is possible, depending on the application, that the sensors operate at different wavelengths with respect to each other.

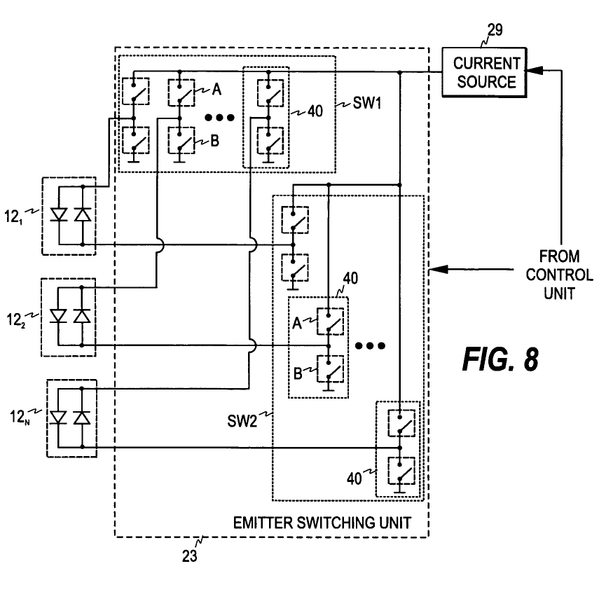

The pulse power supplied to the red emitter elements is typically different from the pulse power supplied to the infrared emitter elements. Therefore, it is advantageous to use a dedicated current source for both emitter element types, as each current source may then use a preset pulse power. However, if the power of a current source may be controlled with sufficient accuracy on a pulse-by-pulse basis, the number of the current sources in the emitter current source may be reduced to one. An embodiment comprising a common current source for all emitter elements of the plurality of sensors is illustrated in FIG. 8. The embodiment shown in the figure corresponds otherwise to the embodiment of FIG. 4, but a single current source 29 connected to both switching unit groups SW1 and SW2 now drives all the emitter elements.

Since at most two current sources are needed for the plurality of sensors operating at one or more wavelengths, the above-described measurement arrangement enables a cost-effective implementation of a monitoring device with multiple sensors. This is due to the fact that the number of current sources (which are relatively expensive) and the number of connection wires may be kept low. Furthermore, no hardware multiplication is needed for the reception side of the monitoring device.

As the present invention may utilize a conventional measurement branch 24 of a single-sensor monitoring device, a conventional monitoring device may be upgraded by providing it with a transmission side capable of connecting each drive pulse to a corresponding emitter element and with a plug-in control software module that enables the device to operate in the time-multiplexed manner described above. The control software module may be delivered, for example, on a data carrier, such as a CD or a memory card, or via a telecommunications network. The software module may be divided into three logical portions according its operation: the first program code portion is configured to control the monitoring device to generate a repeating drive pulse sequence containing drive pulses for all emitter elements of the plurality of sensors, the second program code portion is configured to connect the drive pulses to respective emitter elements in a predetermined order, and the third program code portion is configured to associate an electric reception signal with all emitter elements of the plurality of sensors, one emitter element at a time according to the predetermined order.

Although the invention was described above with reference to the examples shown in the appended drawings, it is obvious that the invention is not limited to these, but may be modified by those skilled in the art without departing from the scope and spirit of the invention. For example, the analysis performed in the control unit on the basis of the measured attenuation may vary according to the application in question. As indicated above, the attenuation may be indicative of the amount of at least one light absorbing substance in the subject. In addition to pulse oximetry, the device may be used, for example, to monitor blood circulation at various tissue sites in connection with blood surgery or to measure the delay associated with the pulsating blood component at various tissue sites.

1. A measurement method for a monitoring device intended for monitoring the attenuation of light in at least one subject, the monitoring device being operably connected to a plurality of sensors, each sensor comprising at least one emitter element for emitting radiation and a sensor-specific detector for receiving the radiation, whereby the monitoring device is operably connected to a plurality of sensor-specific detectors, the method comprising the steps of:
generating a repeating drive pulse sequence containing drive pulses for the emitter elements of the plurality of sensors;
supplying each drive pulse of the drive pulse sequence to a corresponding emitter element;
employing a parallel connection of the plurality of sensor-specific detectors to produce an electric reception signal at a terminal pair common to the sensor-specific detectors; and
receiving the electric reception signal at the monitoring device.

2. A method according to claim 1, wherein each sensor comprises a first emitter element for emitting radiation at a first wavelength and a second emitter element for emitting radiation at a second wavelength, whereby the plurality of sensors comprise a plurality of first emitter elements and a plurality of second emitter elements.

3. A method according to claim 2, wherein the generating step includes the sub-steps of generating the drive pulse sequence by means of a first current source generating drive pulses for the first emitter elements and a second current source generating drive pulses for the second emitter elements.

4. A method according to claim 3, wherein the supplying step includes controlling a first switching element for connecting a drive pulse to the anode of the corresponding emitter element and a second switching element for connecting the cathode of the corresponding emitter element to ground.

5. A method according to claim 1, wherein the receiving step includes connecting the sensor-specific detectors to the monitoring device through a patient cable comprising parallel branches for the plurality of sensors.

6. A method according to claim 1, wherein the generating step includes controlling the power of the drive pulses on a pulse-by-pulse basis.

7. A measurement arrangement for a monitoring device intended for monitoring the attenuation of light in at least one subject, the measurement arrangement comprising:
a plurality of sensors for a plurality of tissue sites, each sensor comprising at least one emitter element for emitting radiation and a sensor-specific detector for receiving the radiation, wherein the sensor-specific detectors are connected in parallel for producing an electric reception signal at a terminal pair common to the sensor-specific detectors;
drive pulse generation means for generating a repeating drive pulse sequence containing drive pulses for the emitter elements of the plurality of sensors;
switching means for connecting each drive pulse of the drive pulse sequence to a corresponding emitter element; and
connection means for connecting the terminal pair operably to the monitoring device, thereby to receive the electric reception signal at the monitoring device.

8. A measurement arrangement according to claim 7, wherein each sensor comprises a first emitter element for emitting radiation at a first wavelength and a second emitter element for emitting radiation at a second wavelength, whereby the plurality of sensors comprise a plurality of first emitter elements and a plurality of second emitter elements.

9. A measurement arrangement according to claim 8, wherein the drive pulse generation means comprise a first current source for generating drive pulses for the first emitter elements and a second current source generating drive pulses for the second emitter elements.

10. A measurement arrangement according to claim 7, wherein the switching means comprise a first group of switching units for the first emitter elements and a second group of switching units for the second emitter elements, each switching unit comprising a first switching element and a second switching element connected in series.

11. A measurement arrangement according to claim 10, wherein
the first current source is operably connected to each first switching element of the first group for connecting each drive pulse to the anode of the corresponding emitter element and the second current source is operably connected to each first switching element of the second group for connecting each drive pulse to the anode of the corresponding emitter element, and
each second switching element is connected to ground for connecting the cathode of the corresponding emitter element to ground.

12. A measurement arrangement according to claim 7, wherein the connection means comprise a patient cable comprising parallel branches for the plurality of sensors.

13. A measurement arrangement according to claim 7, wherein the drive pulse generation means comprise a single current source for generating the drive pulse sequence.

14. A measurement arrangement for a monitoring device intended for monitoring the attenuation of light in at least one subject, the measurement arrangement comprising:
a plurality of sensors for a plurality of tissue sites, each sensor comprising at least one emitter element for emitting radiation and a sensor-specific detector for receiving the radiation, wherein the sensor-specific detectors are connected in parallel for producing an electric reception signal at a terminal pair common to the sensor-specific detectors;
a drive pulse generator unit configured to generate a repeating drive pulse sequence containing drive pulses for the emitter elements of the plurality of sensors;
an emitter switching unit comprising a plurality of switching elements, the emitter switching unit being operably connected to the drive pulse generator unit and to the plurality of sensors thereby to connect each drive pulse of the drive pulse sequence to a corresponding emitter element; and
a measurement cable configured to connect (1) the switching unit to the emitter elements and (2) the terminal pair to the monitoring device thereby to receive the electric reception signal at the monitoring device.

15. A computer program product for controlling a monitoring device intended for monitoring the attenuation of light in at least one subject, the monitoring device comprising a plurality of sensors for a plurality of tissue sites, each sensor comprising at least one emitter element for emitting radiation and a sensor-specific detector for receiving the radiation, the computer program product comprising:
a first program code portion configured to control the monitoring device to generate a repeating drive pulse sequence containing drive pulses for the emitter elements of the plurality of sensors;
a second program code portion configured to connect the drive pulses to respective emitter elements in a predetermined order; and
a third program code portion configured to associate an electric reception signal with the emitter elements of the plurality of sensors, one emitter element at a time according to the predetermined order.
